(19) United States
(12) Patent Application Publication
(10) Pub. No.: US 2021/0205392 A1
(43) Pub. Date: Jul. 8, 2021

(54) Anisomeles indica extract composition for Treating or Improving Gastric Ulcer

(71) Applicant: Syi Biotechnology Co., Ltd., Taichung City (TW)

(72) Inventors: Hsiu-Man Lien, Taichung City (TW); Chia-Chang Chen, Taichung City (TW); Chao-Lu Huang, Taichung City (TW)

(21) Appl. No.: 17/136,597

(22) Filed: Dec. 29, 2020

Related U.S. Application Data
(63) Continuation-in-part of application No. 16/801,825, filed on Feb. 26, 2020.

(30) Foreign Application Priority Data
Jan. 2, 2020 (TW) 109100017

(51) Int. Cl. A61K 36/53; A61P 1/04; A61K 31/365; A61K 31/351

(52) U.S. Cl. CPC A61K 36/53; A61K 31/351; A61K 31/365; A61P 1/04

(57) ABSTRACT

Present invention provides an Anisomeles indica extract comprising ovatodiolide, apigenin-7-glucuronide, acteoside or scutellarin which can be used as an effective ingredient for treating or improving gastric ulcers, including reducing the area of ulcer and inflammation in the stomach tissue.

### BACKGROUND OF THE INVENTION

### 1. Field of the Invention

The present invention relates to the field of gastric ulcers, in particular, a bioactive fraction of Anisomeles indica and active ingredients thereof for treating gastric ulcers.

### 2. Description of the Prior Art

According to statistical data, the prevalence of gastric ulcer disease (PUD) in Europe is 2%, but the rate is as high as 4.7% in Taiwan, which is 2 times higher than that in Europe. Based on the national population, nearly 1 million people in Taiwan may have gastric ulcer. Gastric ulcers tend to emerge gradually and the major pathophysiological progressions of gastric ulcers can develop into the following scenarios: (1) enhanced gastric acid erosion or excessive secretion of gastric juice; (2) mucosal cells cannot be repaired normally; (3) barriers between mucosal cells collapse.

Based on the abovementioned three major pathophysiological progressions, the most common cause of gastric ulcer (or duodenal ulcer) is Helicobacter pylori infection and excessive or inappropriate use of drugs (mainly non-steroidal anti-inflammatory drugs). Nonsteroid anti-inflammatory drugs (NSAID drugs) are commonly used anti-inflammatory and analgesic drugs, of which aspirin can inhibit blood clotting and reduce the occurrence of thrombus, therefore it is often used to prevent the recurrence of myocardial infarction or stroke. NSAID drugs are mainly used to inhibit the catalysis of cyclooxygenase (COX) to achieve the anti-inflammatory, analgesic effects, and antithrombotic effects. However, taking NSAID drugs can cause prohibiting or blocking the repairing process of the gastrointestinal mucosa and subsequently result in gastric ulcer.

At present, common treatments for gastric ulcers include the use of medicines such as antacids to neutralize gastric acid. However, long-term use of antacids brings the risk of extremely low level of the gastric acid and increasing pH of gastric acid, and may also cause overgrow of gastrointestinal bacteria. Liquid antacid is another common drug used to improve gastric ulcers and can attach to the gastric mucosa to form a protective film on the stomach wall, but it is inconvenient to use as it has a shorter effected duration.

Therefore, how to provide an effective ingredient to improve or treat gastric ulcers without side effects is the major topic of this invention.

### SUMMARY OF THE INVENTION

Present invention relates to a method for preparation of a bioactive fraction of Anisomeles indica, TSYI-813, and it is comprising of:

Step 1: using an alcohol solvent for extracting Anisomeles indica for rendering an extract of Anisomeles indica;

Step 2: mixing the extract of Anisomeles indica with an organic solvent and water;

Step 3: purifying an organic layer obtained from Step 2, with the use of a silica gel column chromatography with hexane/ethyl acetate in the ratio of 10:1 to 10:5, wherein the fraction purified with hexane/ethyl acetate in the ratio of 10:5 is the bioactive fraction of Anisomeles indica, TSYI-813.

According to the invention, the extraction temperature of step 1 is 50-80° C.

According to the invention, the extraction time of Step 1 is 4-8 hours.

According to the invention, the ratio of Anisomeles indica and the alcohol solvent used for extraction is 1 (kg): 50-70 (liter).

According to the invention, the alcohol solvent is ethanol.

In another aspect, present invention provides a fraction of Anisomeles indica, which is the bioactive fraction of Anisomeles indica, TSYI-813, prepared by using the method as mentioned above.

In one aspect, present invention provides a method for treating or improving gastric ulcers comprising administering the bioactive fraction of Anisomeles indica, TSYI-813, as mentioned above.

In still another aspect, present invention provides a pharmaceutical composition for treating or improving gastric ulcers, which is comprising of the bioactive fraction of Anisomeles indica, TSYI-813, as mentioned above.

In still another aspect, present invention provides a method for treating or improving gastric ulcers comprising administering an Anisomeles indica extract, wherein the Anisomeles indica extract includes ovatodiolide, apigenin-7-glucuronide (apigenin-7-O-glucuronide), acteoside or scutellarin.

To this end, the Anisomeles indica extract is selected from a combination consisting of ovatodiolide, apigenin-7-glucuronide (apigenin-7-O-glucuronide), acteoside and scutellarin.

Present invention provides a pharmaceutical composition for treating or improving gastric ulcers, which comprises an Anisomeles indica extract with an effective amount, wherein the Anisomeles indica extract includes ovatodiolide, apigenin-7-glucuronide (apigenin-7-O-glucuronide), acteoside or scutellarin.

To this end, the Anisomeles indica extract is selected from a combination consisting of ovatodiolide, apigenin-7-glucuronide (apigenin-7-O-glucuronide), acteoside and scutellarin.

According to the invention, treating or improving gastric ulcers refers to reducing the affected area of gastric ulcer.

According to the invention, treating or improving gastric ulcers refers to reducing the inflammation of stomach tissue.

According to the invention, reducing the inflammation in the stomach tissue refers to reducing the production of prostaglandin E2 (PGE2).

According to the invention, reducing the inflammation in the stomach tissue refers to reducing the production of tumor necrosis factor-α (TNF-α).

According to the invention, said pharmaceutical composition may further include a pharmaceutical acceptable carriers, recipients, diluents, anti-inflammatory agents, or effective ingredients for treating gastric ulcer.

Present invention also provides a food composition which is consisting of the aforementioned bioactive fraction of Anisomeles indica, TSYI-813.

Present invention further provides a food composition which comprises an Anisomeles indica extract wherein the Anisomeles indica extract includes ovatodiolide, apigenin-7-glucuronide (apigenin-7-O-glucuronide), acteoside or scutellarin.

In summary, present invention provides a bioactive fraction of Anisomeles indica, TSYI-813, and a preparation method thereof. The bioactive fraction of Anisomeles indica, TSYI-813, has the effect of treating or improving gastric ulcers and is very suitable for use as an active ingredient for treating gastric ulcers. When used as an active ingredient in the treatment of gastric ulcers, the bioactive fraction of Anisomeles indica, TSYI-813, provided by present invention can not only reduces the area of gastric ulcers, but also reduces the occurrence of gastric inflammation; moreover, the active ingredients, ovatodiolide, apigenin-7-glucuronide (apigenin-7-O-glucuronide), acteoside, and scutellarin, inside the bioactive fraction of Anisomeles indica, TSYI-318, have the effect on treating or improving gastric ulcers and ovatodiolide, which is more effective than other active ingredients displays a curative effect close to omeprazole.

### BRIEF DESCRIPTION OF THE DRAWINGS

The patent or application file contains at least one drawing executed in color. Copies of this patent or patent application publication with color drawing(s) will be provided by the Office upon request and payment of the necessary fee.

FIG. 1 is the flowchart for the gastric ulcer test in mice according to the bioactive fraction of Anisomeles indica, TSYI-813.

FIG. 2 is the flowchart for the gastric ulcer test in mice according to all active ingredients of TSYI-813.

FIG. 3A shows the effect of the bioactive fraction of Anisomeles indica, TSYI-813, based on the changes of the body weights of mice in the gastric ulcer test; showing the changes in the weight of the test mice from Day 0 (D0) to Day 24 (D24).

FIG. 3B shows the effect of the bioactive fraction of Anisomeles indica, TSYI-813, based on the changes of the body weights of mice in the gastric ulcer test; showing the changes in the weight of the test mice from Day 0 (D0) to Day 37 (D37).

FIG. 4 shows the effects of all active ingredients of TSYI-813 on changes in the weight of test mice.

FIG. 5 shows the effect of the bioactive fraction of Anisomeles indica, TSYI-813, on gastric ulcer based on the changes in the affected area of gastric ulcer in the test mice.

FIG. 6 shows the effects of all active ingredients of TSYI-813 on changes in gastric ulcer areas of test mice.

FIG. 7 shows the effect of the bioactive fraction of Anisomeles indica, TSYI-813, on the ulcer index in the test mice;

FIG. 8 shows the effects of all active ingredients of TSYI-813 on changes in the ulcer index of test mice.

FIG. 9 shows the effect of the bioactive fraction of Anisomeles indica, TSYI-813, on gastric pathology in the test mice;

FIG. 10 shows the effects of all active ingredients of TSYI-813 on pathological conditions of stomachs in test mice.

FIG. 11 shows the effect of the bioactive fraction of Anisomeles indica, TSYI-813, on the TNF-α level in the stomach tissue of the test mice;

FIG. 12 shows the effect of the bioactive fraction of Anisomeles indica, TSYI-813, on the PGE2 level in the stomach tissue of the test mice;

### DETAILED DESCRIPTION OF THE PREFERRED EMBODIMENT

Unless defined otherwise, all technical and scientific terms described in this specification have the meaning commonly understood by those skilled in the art.

The singular terms “a”, “an” and “the” as used in this specification and the scope of the patent application may refer to more than one subject unless otherwise stated.

“Or”, “and”, and “and” used in this specification refer to “or/and” unless stated otherwise. In addition, the terms “including” and “comprising” are open-ended connectives without restrictions. The preceding paragraph is a systematic reference only and should not be construed as a limitation on the subject of the invention.

The terms “treating”, “for treatment” and the like refer to methods of delaying, ameliorating, reducing, or reversing a diagnosable condition suffered by a patient and the associated symptoms caused by the condition, and the methods for prevention of the condition or any related symptoms.

The term “pharmaceutically acceptable” refers to that the substance or composition must be compatible with the other ingredients of its pharmacological formulation without exacerbating the symptoms of the patient.

The composition provided by the present invention can be prepared by using technologies well known to those having ordinary knowledge in the technical field to which the present invention belongs and is prepared by combining the active ingredient or composition provided in the present invention with at least one pharmaceutically acceptable vehicle. A dosage form suitable for the composition of the present invention. The dosage forms include, but are not limited to, solutions, emulsions, suspensions, powders, lozenges, lozenges, tablets, chewing gums, capsules, and other similar or applicable dosage forms of the present invention.

The term “pharmaceutically acceptable carrier” includes one or more types of ingredients selected from the group consisting of solvents, emulsifiers, suspending agents, disintegrating agents, binding agents, excipients, stabilizers, chelating agents, diluents, gelling agents, preservatives, lubricants, surfactants, and other carriers similar or suitable for use in the present invention.

To the aforementioned composition, one or more of the abovementioned dissolution aids, buffering agents, coloring agents, flavoring agents and the like, which are generally used in the formulation field, may also be appropriately added as needed.

The term “pharmaceutically acceptable excipients” include, but are not limited to, at least one of the following: polymers, resins, plasticizers, fillers, lubricants, diluents, binders, disintegrants, solvents, co-solvents, surfactants, preservatives, sweeteners, flavoring agents, pharmaceutical-grade dyes or pigments and viscosity modifiers.

The term “pharmaceutical composition” refers to a solid or liquid composition in a form, concentration, and degree of purity suitable for administration to a patient. After administration, it can induce desired physiological changes; the pharmaceutical composition is sterile and/or non-pyrogenic.

The term “effective amount” refers to the amount necessary to produce and cause an expected response in the body, and is not a quantity required for therapeutic recovery. Those of ordinary skill in the art to which this invention pertains will understand that the effective amount of a pharmaceutical composition may vary depending on factors such as the desired biological endpoint, the bioactive agent to be delivered, the composition of the encapsulating matrix and the target tissue, etc.

The effective amount of the bioactive fraction of Anisomeles indica, TSYI-813, in the human body can be calculated based on the effective amount in the mice provided in the examples of this invention by using the differences in the body surface area (the conversion factor for mouse and human is 12.3-fold) and the formulas proposed by the USFDA: if the effective amount of mice is 5.2 mg/kg BW/day, then the effective amount of 40 kg human body is 5.2/12.3×40=16.9 mg/day; if the effective amount of mice is 10.4 mg/kg BW/day, the effective amount of 100 kg human body is 10.4/12.3×100=84.6 mg.

The effective amount of each active ingredient of TSYI-813 in the human body can be calculated based on the effective amount in mice provided in examples of this invention by using the differences in the body surface area (the conversion factor for mouse and human is 12.3-fold) and the formulas proposed by the USFDA: if the effective amount of mice is 10 mg/kg BW/day, then the effective amount of the human body is 10/12.3=0.813 mg/kg BW/day; accordingly, the effective amount of the 40 kg human body is 10/12.3×40=32.5 mg/day and the effective amount of the 100 kg human body is 10/12.3×100=81.3 mg

Unless otherwise specified, the materials used in the present invention are commercially available materials. Anisomeles indica (L.) K t z e used in the examples of the present invention can be purchased or collected in the wild. (L.) Present invention uses Anisomeles indica (L.) Ktze as the example, but all plants of the genus Anisomeles should be included in the present invention.

The test animals to be checked with the bioactive fraction of Anisomeles indica, TSYI-813, in the embodiment of the present invention are 8-week-old male specific pathogen free (SPF) C57BL/6 strain mice, which were purchased from BioLASCO Taiwan Co., Ltd; the test animals to be checked with active ingredients of TSYI-813 are 8-week-old male specific pathogen free (SPF) ICR strain mice, which were purchased from BioLASCO Taiwan Co., Ltd.

The present invention will be better elucidated when read in conjunction with the following examples; however, it should be understood that the invention is not limited to the preferred embodiments shown.

### Example 1: Preparation of the Bioactive Fraction of Anisomeles indica, TSYI-813

Following steps are used for preparation of the bioactive fraction of Anisomeles indica, TSYI-813

Step 1: using an alcohol solvent to extract Anisomeles indica (L.) Ktze (dried whole plant) at a certain temperature for a certain time period to give an extract of Anisomeles indica; wherein the ratio of said Anisomeles indica (L.) Ktze and the alcohol solvent is 1 (kg):50˜70 (liter), and a preferred ratio is 1 (kg):60 (liter); wherein the alcohol solvent includes, but is not limited to, methanol, ethanol, propanol, butanol, and a preferred alcohol solvent is ethanol; wherein the volume percentage concentration of the alcohol solvent is 65-85% and an optimal concentration is 75%; wherein the certain temperature is ranging from 50° C. to 80° C., and a preferred temperature is 70° C.; wherein the certain time period is 4-8 hours, and a preferred time period is 6 hours.

Step 2: Concentrating the extract of Anisomeles indica and then further extracting the concentrated extract by using an organic solvent and water to give an organic solvent layer and an aqueous layer, among which the organic solvent is comprising of, but is not limited to, phenol and chloroform, and the preferred organic solvent is chloroform.

Step 3: concentrating the organic solvent layer from Step 2, and then purifying the concentrated organic solvent layer by a silica gel column chromatography (Silica gel 60 Å, 230-400 mesh). More specifically, sequentially eluting the concentrated organic solvent layer using the silica gel column chromatography with hexane/ethyl acetate (ratio=10:1, 10:2, 10:3, 10:4 and 10:5 in turn), and collecting the fraction eluted with hexane/ethyl acetate (ratio=10:5) to give the bioactive fraction of Anisomeles indica, TSYI-813, (hereinafter called TSYI-813). Next, TSYI-813 was further concentrated and freeze-dried to give a lyophilized powder for following tests. One kg of the whole plant of Anisomeles indica can give 705 mg of the lyophilized powder of TSYI-813.

### Example 2: Analysis of Active Ingredients of TSYI-813

The active ingredients in the TSYI-813 were determined by the high performance liquid chromatography (HPLC) according to the practical analysis method as follows: 20 mg of lyophilized powder of TSYI-813 were dissolved with a methyl alcohol in a 10 ml volumetric flask; after quantified, the dissolved TSYI-813 was filtered with a 0.45 μm filter membrane and then analyzed by HPLC, compared with standard solutions.

It can be seen from analysis results that the active ingredients of TSYI-813 include ovatodiolide, apigenin-7-glucuronide, acteoside and scutellarin. The content of each active ingredient in 1 g lyophilized powder of TSYI-813 is shown in Table 1.

TABLE 1

| Active ingredient | Content | |
|---|---|---|
| Ovatodiolide | 69.2 | mg/g |
| Acteoside | 64.3 | mg/g |
| Scutellarin | 6.9 | mg/g |
| Apigenin-7-glucuronide | 170 | mg/g |

The chemical structural formula of each active ingredient is shown as follows:

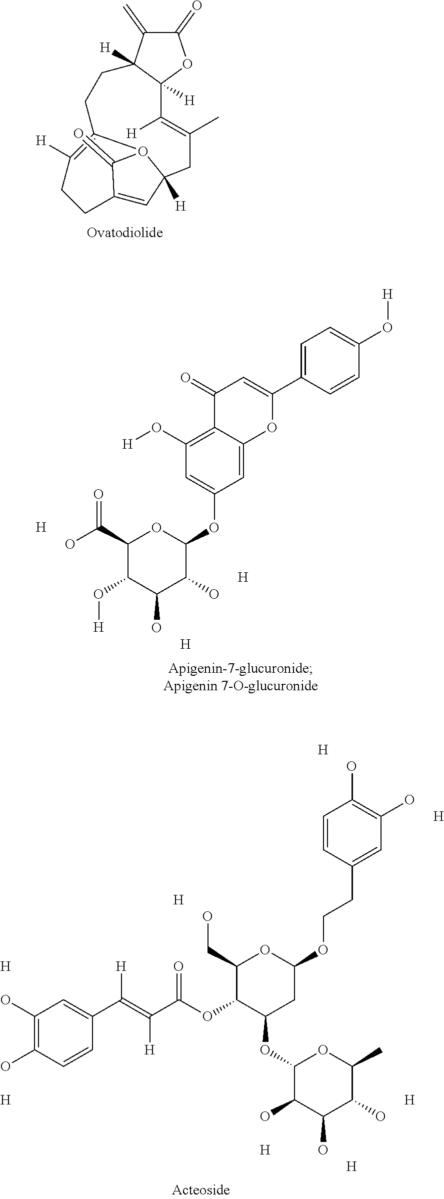

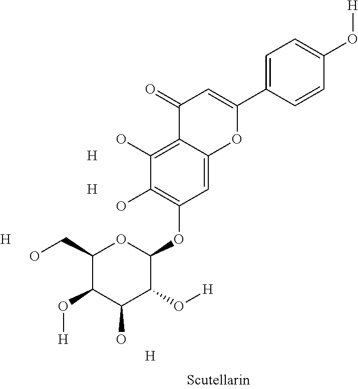

### Example 3: Analysis of the Effects of TSYI-813 and Active Ingredients Thereof on Treating Gastric Ulcer

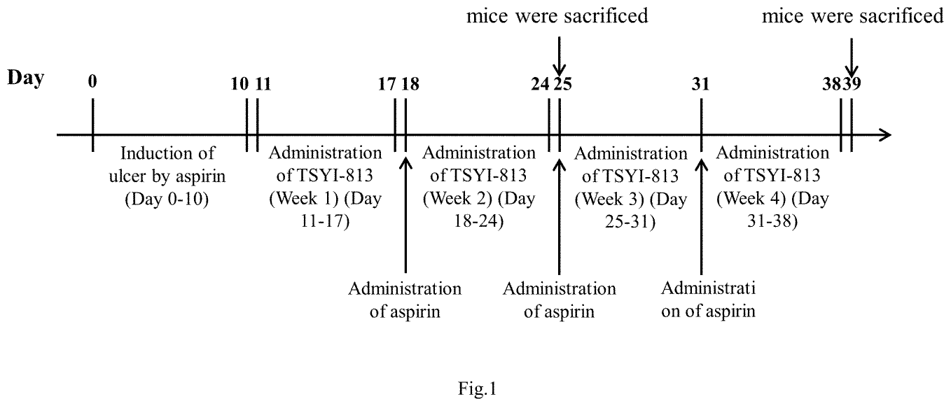

Experimental process of TSYI-318 for gastric ulcers in mice: As shown in FIG. 1, the test mice were given aspirin, 500 mg per kg of weight (500 mg/kg BW), for 10 days to induce gastric ulcer. Next, the test mice were given drinking water or TSYI-813 via gavage for 4 weeks, and the dose are as follows:

Negative control group: 0.1 mL drinking water per day;

TSYI-813 low-dose group: 5.2 mg per body weight per day (5.2 mg/kg BW);

TSYI-813 middle-dose group: 10.4 mg per body weight per day (10.4 mg/kg BW).

The gastric ulcer test in mice was continued for 38 days starting from the first administration of aspirin. During the test period, the mice were weighed and a dose of the test sample (TSYI-813) corresponding to the body weight of the mice was given every day. Two weeks and four weeks after administration of TSYI-813, half of the test mice in each group were sacrificed to observe the condition of gastric ulcer in their stomach. During the test period, aspirin was given in the amount of 500 mg per kg of body weight (500 mg/kg BW) to the test mice once a week to maintain the lesion of gastric ulcer in the test mice.

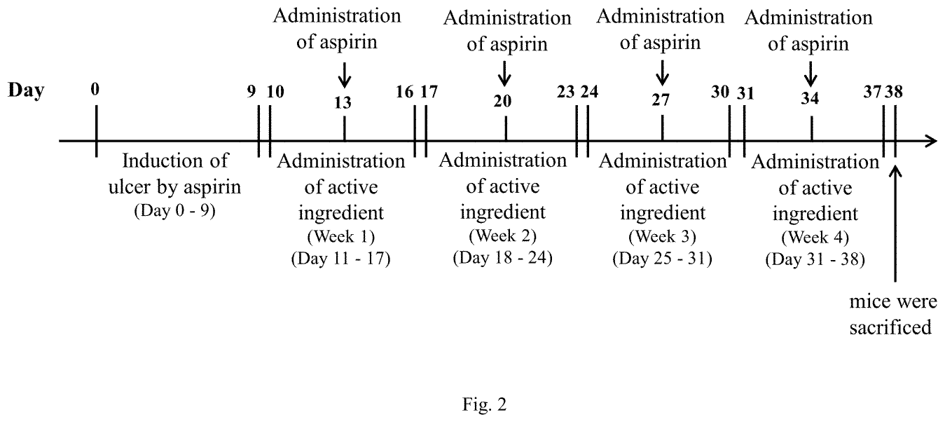

Experimental process of all active ingredients of TSYI-318 for gastric ulcers in mice: As shown in FIG. 2, the test mice of all groups except those of the normal control group were fed with aspirin, 500 mg per kg of body weight (500 mg/kg BW), for 10 days to induce gastric ulcers. After the inductive phase, these mice were fed with aspirin (500 mg/kg BW) once a week for keeping the symptom of gastric ulcer. Moreover, these mice were fed with drinking water, the standard drug (omeprazole) or one active ingredient of TSYI-813, each of which was included in an oral gavage, for 4 weeks after the inductive phase; these mice were sacrificed for following analyses on Day 38. During the test period, the mice were weighed and fed with the corresponding doses of standard drugs or active ingredients of TSYI-813 (0.1 mL-0.2 mL in total) everyday based on their body weights, as shown below:

Normal control group (no aspirin fed): 0.1 mL drinking water per day;

Positive control group: Omeprazole (10 mg/kg BW) per day;

Negative control group: 0.1 mL drinking water per day;

AI-C1 group: Ovatodiolide (10 mg/kg BW) per day;

AI-C2 group: Apigenin-7-glucuronide (10 mg/kg BW) per day;

AI-C3 group: Acteoside (10 mg/kg BW) per day;

AI-C4 group: Scutellarin (10 mg/kg BW) per day.

Pathological Analysis of Stomach Tissue:

The image analysis software (Image J) was used to identify the ulcer lesions in the stomach tissue to calculate the ulcer area of the stomach of each test mouse, and the grade of ulcer was divided into three levels according to the area of ulcer. Next, calculate the ulcer index (UI) and curative ratios (%) according to Table 2 and the following formulas:

UI=[(1×number of level I)+(2×number of level II)+(3×number of level III)]÷number of mice in each group

Curative ratio (%)=100−(UI of test group÷UI of control group)×100

TABLE 2
Classification and scoring of ulcer area

| Level | level I | level II | level III |
|---|---|---|---|
| Area | <1 mm2 | 1~3 mm2 | >3 mm2 |
| Score | 1 | 2 | 3 |

Test Results of the Effect of TSYI-348 on Mice's Gastric Ulcers:

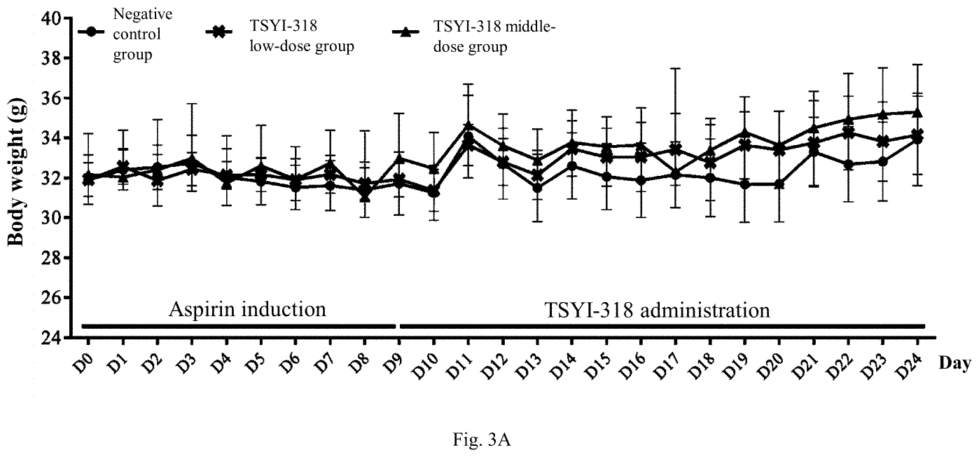

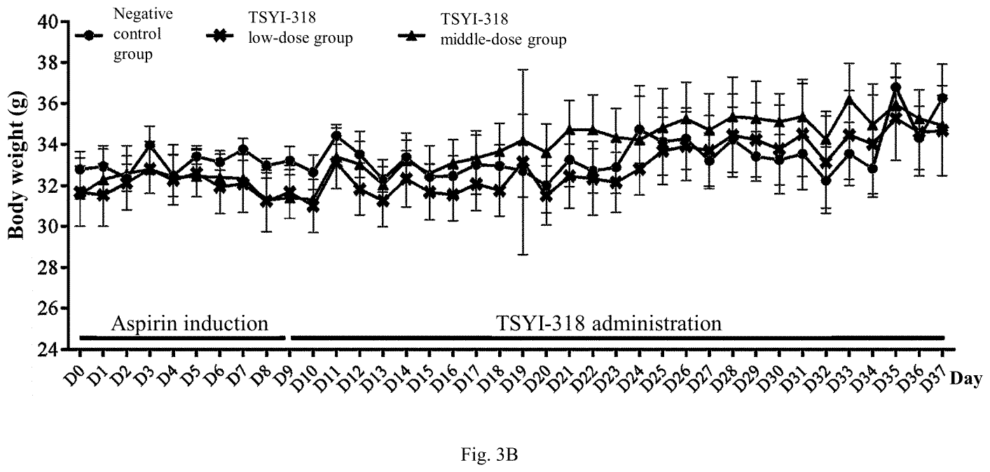

The weight change of the test mice is shown in FIGS. 3A & 3B. The weight change between the groups is not obvious. After the mice of each group were given TSYI-813, their weight began to increase, although the average weight of the negative control group was slightly higher than TSYI-813 low-dose group and TSYI-813 middle-dose group, but there was no significant difference between that of the groups according to the statistical analysis.

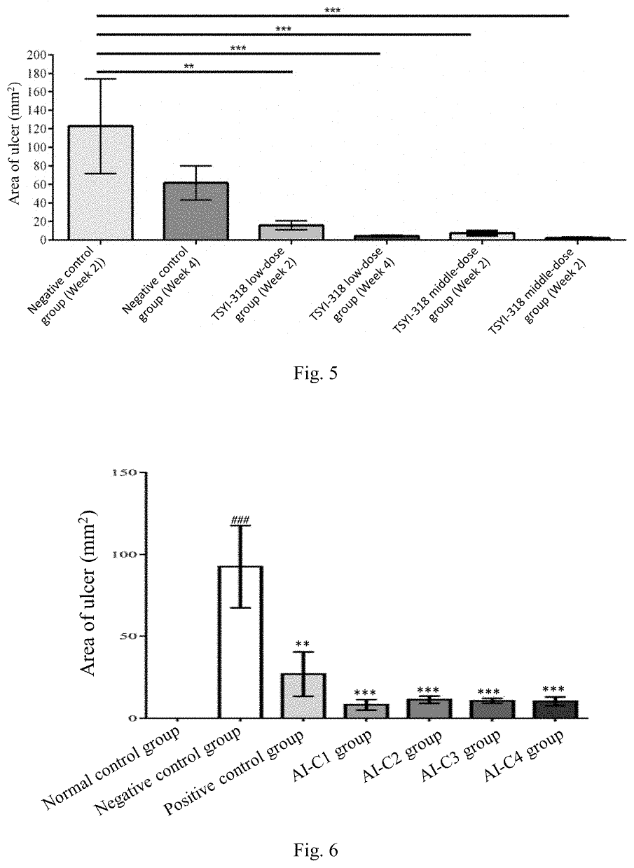

The change in the area of gastric ulcer is shown in FIG. 5. In Week 2 and Week 4 after TSYI-813 administration, the areas of gastric ulcer in the mice of TSYI-318 low-dose group or the TSYI-318 meddle-dose group were all smaller than that in the mice of the negative control group. This result shows that TSYI-813 has the effect of improving the area of ulcer and this effect is dose dependent.

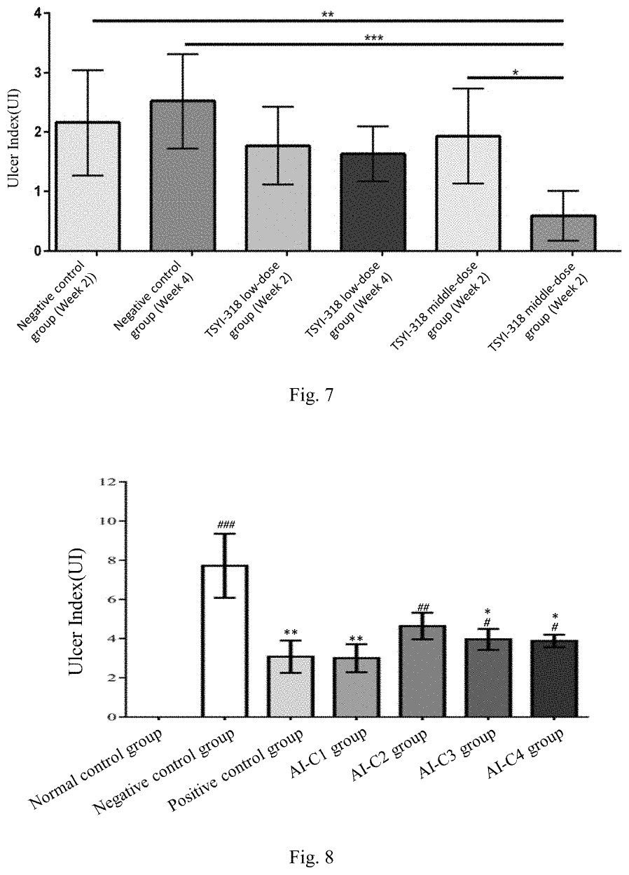

The calculated results of UI and curative ratio are shown in FIG. 7 and Table 3. TSYI-318 low-dose group and TSYI-318 middle-dose group, after 4 weeks of administration of TSYI-813, their UI were all lower than that of the negative control group (FIG. 7). In addition, after administration of TSYI-813 for 4 weeks, the curative ratio of the TSYI-318 low-dose group is 67.12% (Table 3). Such results indicate that TSYI-813 has the effect of treating gastric ulcer.

TABLE 3

Curative ratio (%)

| | Week 2 | Week 4 |
|---|---|---|
| TSYI-318 low-dose group | −0.84 | 9.30 |
| TSYI-318 middle-dose group | −13.45 | 67.12 |

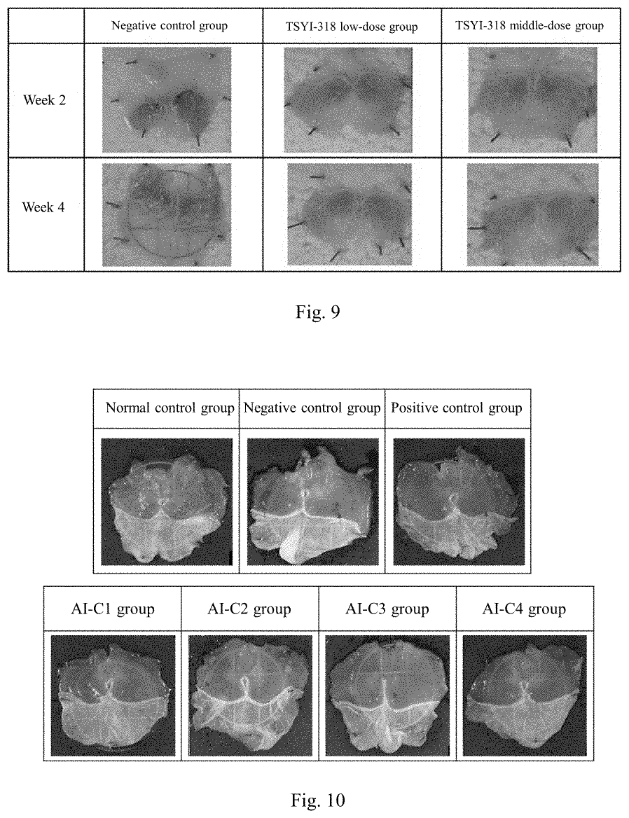

The pathological conditions of the stomach are shown in FIG. 9. Observation of the gastric mucosal surface of the mice shows that aspirin can induce needle-like bleeding points and different sizes of ulcer lesion areas on the gastric mucosal surface of the test mice and most of the ulcer lesions appear randomly and scattered in the gastric glands. After analyzing the ulcer lesions of the test mice by image analysis software, it was found that the administration of low or meddle doses of TSYI-813 can improve the ulcer of the test mice (Table 4).

TABLE 4

Ulcer area (mm²)

| | Week 2 | Week 4 |
|---|---|---|
| Negative control group | 122.90 ± 51.26 | 61.55 ± 18.36 |
| TSYI-318 low-dose group | 15.80 ± 4.82 | 4.19 ± 0.66 |
| TSYI-318 middle-dose group | 7.34 ± 2.48 | 2.07 ± 0.74 |

Test Results of the Effects of Active Ingredients in TSYI-348 on Mice's Gastric Ulcers:

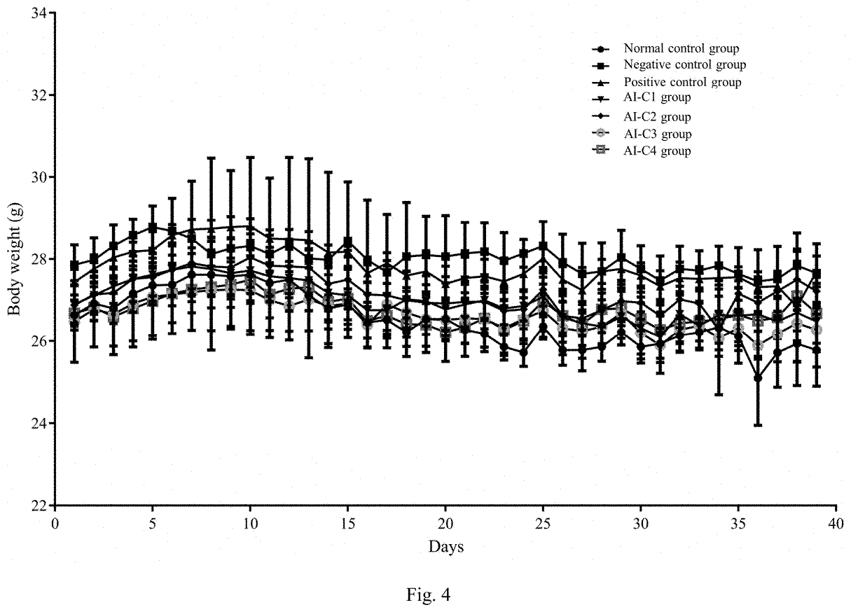

As shown in FIG. 4, the changes in weights of test mice between groups show no significant difference that means no discomfort is observed on test mice fed with the standard drug (omeprazole) or each active ingredient of TSYI-813 (ovatodiolide, apigenin-7-glucuronide, acteoside and scutellarin) (p>0.05).

The pathological conditions of stomach are shown in FIG. 10. Gastric ulcers induced by aspirin (500 mg/kg BW) are observed on gastric mucosal surfaces of mice, for example, needle-like bleeding points and different sizes of superficial ulcer lesion areas on gastric mucosal surfaces of test mice, and most ulcer lesions appear randomly and are scattered in the gastric glands (as shown in the arrow for the ulcer lesion).

The changes in the areas of gastric ulcers are shown in FIG. 6 and Table 5: the areas of gastric ulcers in the negative control group are significantly higher than those in other groups; the areas of gastric ulcers in each group fed with active ingredients of TSYI-318 are smaller than those in the negative control group and the positive control group. It can be seen from test results that all active ingredients of TSYI-318 contribute to reducing the areas of gastric ulcers and the optimal effect on reducing the areas of gastric ulcers is observed on mice fed with ovatodiolide (AI-C1 group).

TABLE 5

| | Ulcer area (mm²) |
|---|---|
| Normal control group | 0 ± 0 |
| Negative control group | 92.63 ± 25.04^{###} |
| Positive control group | 26.90 ± 13.52** |
| AI-C1 group (mice fed with ovatodiolide) | 8.22 ± 3.23*** |
| AI-C2 group (mice fed with apigenin-7-glucuronide) | 11.34 ± 2.20*** |
| AI-C3 group (mice fed with acteoside) | 10.66 ± 1.50*** |
| AI-C4 group (mice fed with scutellarin) | 10.36 ± 2.60*** |

Data is represented by “mean ± SEM”.
#: normal control group vs. negative control group;
* negative control group vs. positive control group (AI-C1 group, AI-C2 group, AI-C3 group or AI-C4 group).
(#, * p < 0.05; ##, **p < 0.01; ^{###}, ***p < 0.001)

As shown in FIG. 8 and Table 6 for calculations of the ulcer index, the ulcer index of the negative control group is significantly higher than that of the positive control group (p<0.01), the AI-C1 group (p<0.01), the AI-C3 group (p<0.05) and the AI-C4 group (p<0.05); the significant difference is observed between the normal control group and the negative control group (p<0.001), between the normal control group and the AI-C2 group (p<0.01), between the normal control group and the AI-C3 group (p<0.05) and between the normal control group and the AI-C4 group (p<0.05).

TABLE 6

| | Ulcer index |
|---|---|
| Normal control group | 0 ± 0 |
| Negative control group | 7.84 ± 1.66^{###} |
| Positive control group | 3.08 ± 0.83** |
| AI-C1 group (mice fed with ovatodiolide) | 3.00 ± 0.72** |
| AI-C2 group (mice fed with apigenin-7-glucuronide) | 4.64 ± 0.68^{##} |
| AI-C3 group (mice fed with acteoside) | 3.96 ± 0.53^{#}* |
| AI-C4 group (mice fed with scutellarin) | 3.88 ± 0.32^{#}* |

Data is represented by “mean ± SEM”.
^{#}normal control group vs. negative control group (AI-C2 group, AI-C3 group or AI-C4 group);
*negative control group vs. positive control group (AI-C1 group, AI-C3 group or AI-C4 group).
(^{#}, *p < 0.05; ^{##}, **p < 0.01; ^{###}, *** p < 0.001)

As shown in Table 7 for calculated curative ratios of test mice fed with the standard drug (omeprazole) and active ingredients of TSYI-318 for 4 weeks, the curative ratios of the positive control group (omeprazole), the AI-C1 group (ovatodiolide), the AI-C2 group (apigenin-7-glucuronide), the AI-C3 group (acteoside) and the AI-C4 group (scutellarin) are 60.71%, 61.73%, 39.90%, 48.70% and 49.74%, respectively.

It can be seen from above test results that all active ingredients of TSYI-318 display the curative effect on gastric ulcers and the order of active ingredients based on the curative ratios from high to low is ovatodiolide, scutellarin, acteoside and apigenin-7-glucuronide.

TABLE 7

| | Curative ratio(%) |
|---|---|
| Positive control group | 60.71% |
| AI-C1 group (mice fed with ovatodiolide) | 61.73% |
| AI-C2 group (mice fed with apigenin-7-glucuronide) | 39.90% |
| AI-C5 group (mice fed with acteoside) | 48.70% |
| AI-C4 group (mice fed with scutellarin) | 49.74% |

### Example 4: Analysis of the Effect of Bioactive Fraction of Anisomeles indica, TSYI-813 on Biochemical Indexes of Stomach Tissue

At Week 2 and Week 4 after TSYI-813 administration, the mice of each group were sacrificed and the stomach tissues were homogenized at low temperature. After centrifugation, the supernatant was analyzed for biochemical indicators related to inflammation such as prostaglandin E2 (PGE2), tumor necrosis factor-α. (TNF-α) and total protein content.

Prostaglandin E 2 (PGE2) Level Analysis:

The competitive-ELISA was used to analyze the PGE2 level in stomach tissue. The test sample (the aforementioned supernatant) and a known concentration of PGE2 (PGE2 standard) were added to a 96-well microtiter plate pre-coated with mouse PGE2 antibody for reaction at 37° C. for 45 minutes, so as to allow the PGE2 or PGE2 standard in the sample competes for the PGE2 antibody binding site on the microtiter plate. Next, the excess, unbound sample or PGE2 standard was removed from the microtiter plate, and then avidin-peroxidase (avidin-HRP) was added for reaction at 37° C. for 45 minutes. Next, 3,3′, 5,5′-Tetramethylbenzidine (TMB) was added for 15 minutes to allow color reaction. After the reaction was stopped, the plate was analyzed by measuring the absorbance at 450 nm with a microplate reader. Finally, the absorbance of each sample was compared with the PGE2 standard curve to calculate the PGE2 concentration of each sample.

Analysis of Tumor Necrosis Factor-α (TNF-α) Level:

Sandwich-ELISA was used to analyze the TNF-α level in stomach tissues. The test sample (the aforementioned supernatant) and a known concentration of TNF-α (TNF-α standard) were added to a 96-well microtiter plate pre-coated with mouse TNF-α antibody and reacted at 37° C. for 90 minutes to allow binding of the TNF-α or TNF-α standard in the sample to the antibody. Then the mouse TNF-α antibody conjugated with avidin was added and allowed to react at 37° C. for 1 hour and the excess, unbound sample or TNF-α antibody was removed from the microtiter plate before the avidin-peroxidase (avidin-HRP) was added and incubated at 37° C. for 30 minutes; then 3,3 ‘, 5,5’-tetramethylbenzidine (TMB) was added for 15 minutes to allow color reaction. After stopping the reaction, the plate was analyzed by measuring the absorbance at 450 nm with a microplate reader. Finally, the absorbance of each sample was compared with the TNF-α standard curve to calculate the TNF-α concentration of each sample.

Total Protein Content:

Bradford analysis was used to quantify the total protein content of the stomach tissue. Specifically, 1 mL of a commercially available Bradford reagent was added to a known concentration of protein standard solution (0, 25, 50, 75, and 100 ug/mL) or to 0.2 mL aforementioned supernatant (250-fold dilution), after reacting at room temperature for 2 minutes, the absorbance at 595 nm was measured and the absorbance of each sample was compared with the protein standard curve to calculate the concentration of the total protein of each sample.

The Results of Biochemical Indicators Analysis in Stomach Tissue

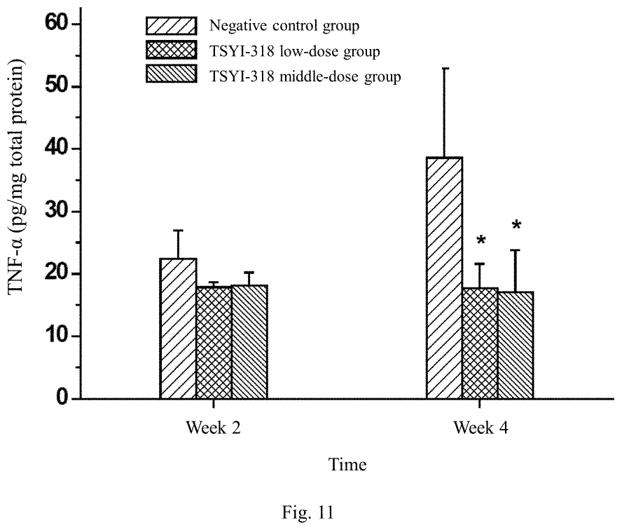

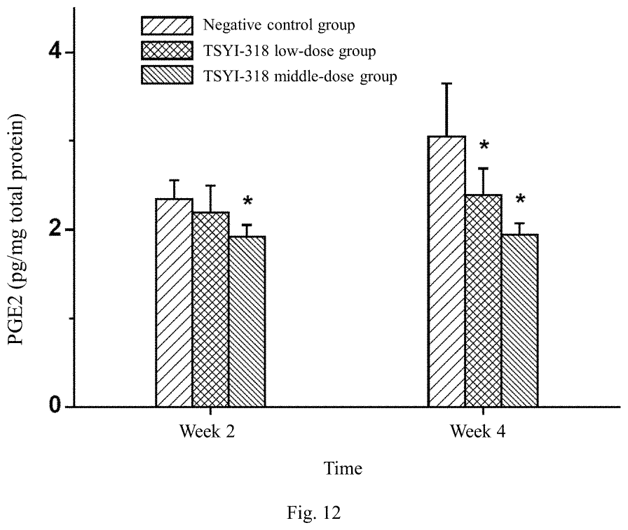

The results are shown in FIG. 11, FIG. 12 and Table 8. After administration of TSYI-813 to test mice, whether at low or meddle doses, the TNF-α level in the gastric tissue of the test mice was significantly lower than that of the negative control group (FIG. 11) at Week 4. Similar results were also found in the prostaglandin 2 (PGE2). After administration of TSYI-813 at a middle dose to the test mice, PGE2 production was significantly inhibited at Week 2 and Week 4, while the production of PGE2 in the TSYI-318 low-dose group was also significantly inhibited at Week 4, and the inhibition was dose-dependent (FIG. 12). These results show that TSYI-813 can reduce the inflammation of stomach tissues, and the results indicate that TSYI-813 can inhibit inflammation in stomach tissue, thereby achieving the effect of treating gastric ulcers.

TABLE 8

Inhibition (%)

| | | Week 2 | Week 4 |
|---|---|---|---|
| TNF-α | TSYI-318 low-dose group | 20.2 | 55.2 |
| | TSYI-318 middle-dose group | 19.2 | 56.8 |
| PGE2 | TSYI-318 low-dose group | 6.5 | 21.7 |
| | TSYI-318 middle-dose group | 18.1 | 36.3 |

According to the results mentioned above, TSYI-813 at low or middle doses has the effect of treating gastric ulcer, and the effect of 4-week TSYI-813 treatment is better than 2 weeks of TSYI-813 treatment; in addition, middle-dose TSYI-318 treatment has a better curative rate for gastric ulcer. The results of the above examples can also confirm that the bioactive fraction of Anisomeles indica, TSYI-813, provided by the present invention can be used as an active ingredient for treating or improving gastric ulcer and exhibits the advantages of inhibiting the production of PGE2 and TNF-α without affecting body weight. Furthermore, it can be seen from test results that each of all active ingredients in TSYI-318 including ovatodiolide, scutellarin, acteoside and apigenin-7-glucuronide displays the curative effect on gastric ulcer and the optimal effect is observed at mice fed with ovatodiolide and close to that of mice fed with ovatodiolide.

Many changes and modifications in the above described embodiment of the invention can, of course, be carried out without departing from the scope thereof. Accordingly, to promote the progress in science and the useful arts, the invention is disclosed and is intended to be limited only by the scope of the appended claims.

What is claimed is:

1. A pharmaceutical composition for treating or improving gastric ulcers, wherein the pharmaceutical composition comprising an effective amount of an Anisomeles indica extract including ovatodiolide, apigenin-7-glucuronide, acteoside or scutellarin.

2. The pharmaceutical composition as recited in claim 1, wherein the treating or improving gastric ulcer refers to reducing the area of gastric ulcer.

3. The pharmaceutical composition as recited in claim 1, wherein the treating or improving gastric ulcer refers to reducing inflammation in the stomach tissue.

4. The pharmaceutical composition as recited in claim 3, wherein the reducing inflammation in stomach tissue refers to reducing the production of prostaglandin E2 (PGE2).

5. The pharmaceutical composition as recited in claim 3, wherein the reducing inflammation in stomach tissue refers to reducing the production of tumor necrosis factor-α (TNF-α).

6. The pharmaceutical composition as recited in claim 1, wherein the pharmaceutical composition may further comprise pharmaceutically acceptable carriers, recipients, diluents, anti-inflammatory agents or effective ingredients for treating gastric ulcers.

7. A method for treating or improving gastric ulcers comprising administering to a subject in need thereof an effective amount of a pharmaceutical composition as recited in claim 1, wherein the subject in need thereof suffers from gastric ulcers.

8. The method as recited in claim 7, wherein the treating or improving gastric ulcer refers to reducing the area of gastric ulcer.

9. The method as recited in claim 7, wherein the treating or improving gastric ulcer refers to reducing inflammation in the stomach tissue.

10. The method as recited in claim 9, wherein the reducing inflammation in stomach tissue refers to reducing the production of prostaglandin E2 (PGE2).

11. The method as recited in claim 9, wherein the reducing inflammation in stomach tissue refers to reducing the production of tumor necrosis factor-α (TNF-α).
